(19) United States
(12) Patent Application Publication
(10) Pub. No.: US 2014/0222009 A1
(43) Pub. Date: Aug. 7, 2014

(54) ASSEMBLY FOR KYPHOPLASTY PROCEDURES

(71) Applicant: Angelo Piras, Roma (IT)

(72) Inventor: Angelo Piras, Roma (IT)

(73) Assignee: O.P.A. MEDICAL. S.R.L., ROMA (IT)

(21) Appl. No.: 14/343,541

(22) PCT Filed: Aug. 14, 2012
(86) PCT No.: PCT/IB2012/054140

§ 371 Date: Mar. 7, 2014

(30) Foreign Application Priority Data
Sep. 9, 2011 (IT) RM2011A000479

(51) Int. Cl. A61B 17/88

(52) U.S. Cl. CPC A61B 17/8855
USPC 606/94

(57) ABSTRACT

An assembly for kyphoplasty procedures (1) comprising a syringe (5), which is suited to inflate a balloon inserted into a cavity obtained in the vertebral column, and an injector (6), which is suited to introduce cement into the vertebral cavity formed. The assembly for kyphoplasty procedures comprises, furthermore, a control unit (2) and a pair of motors (7, 8), each of which is suited to move a respective plunger (9, 10) belonging to the syringe (5) and to the injector (6) respectively. The motors (7, 8) are connected to the control unit (2).

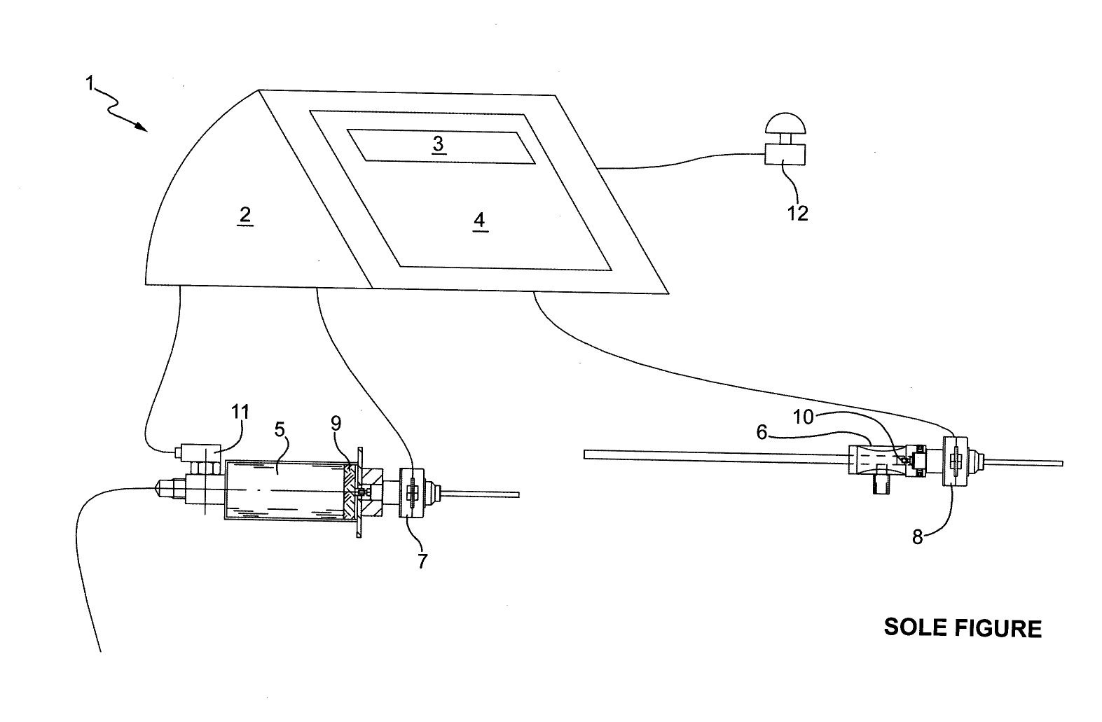

### TECHNICAL FIELD

The present invention is relative to an assembly for kyphoplasty procedures.

### BACKGROUND ART

Kyphoplasty is a technique that, for many years now, has been used to treat spinal fractures. Substantially, kyphoplasty involves the introduction of a balloon into the vertebral column, or into a bone in general, in correspondence to the fracture; said balloon is inflated, thus re-establishing the correct vertebral arrangement; subsequently, the balloon is deflated and removed, so as to fill the cavity created by the balloon inflated with a biocompatible cement, in order to make sure that the vertebral arrangement, which has been re-established, is maintained.

The introduction and the inflation of the balloon, as well as the insertion of the cement, are performed by means of suited syringes.

Obviously, this technique has to be supported by a series of safety systems, so as to prevent iatrogenic complications from occurring. Indeed, even the smallest mistake can compromise the motor skills of the patient.

In particular, kyphoplasty procedures have to be carefully monitored during the creation of the vertebral cavity by means of the balloon and during the filling of the cavity with the cement.

A monitoring system used is relative to radiography imaging techniques, which allow medical personnel to perform kyphoplasty procedures with high degrees of safety.

Even though the use of radiography imaging techniques fully fulfils the safety needs mentioned above, it is affected by the problem of exposing the medical personnel to ionizing radiations.

For this reason, the medical personnel involved is provided with individual protections, which, though, due to comfort and freedom of movement reasons, are used in a partial way or not used at all.

### DISCLOSURE OF INVENTION

The object of the prevent invention is to provide an assembly for kyphoplasty procedures, whose technical features are such as to guarantee a correct monitoring of the different steps of the procedure, thus preventing at the same time the medical personnel involved from being exposed to the action of ionizing radiations.

The subject-matter of the present invention is an assembly for kyphoplasty procedures, whose essential features are set forth in claim 1, and whose preferred and/or auxiliary features are set forth in claims 2-7.

### BRIEF DESCRIPTION OF THE DRAWING

The present invention will be best understood upon perusal of the following detailed description of an illustrative and non-limiting embodiment with reference to the accompanying FIGURE, which shows a schematic view, with some parts removed, of an assembly for kyphoplasty procedures according to the present invention.

### BEST MODE FOR CARRYING OUT THE INVENTION

In the FIGURE, number 1 indicates an assembly for kyphoplasty procedures according to the present invention as a whole.

The assembly 1 comprises a control unit 2, a display 3, which is connected to the control unit 2 and is suited to transmit information to the medical personnel, and a keyboard 4, through which the medical personnel communicates with the control unit 2, so as to set the operations to be performed.

The assembly 1 comprises, furthermore, a syringe 5, which is useful to inflate the balloon (which is not illustrated for simplicity reasons), and an injector 6, which is useful to introduce the cement into the vertebral cavity created. The syringe 5 and the injector 6 are associated to a respective stepper motor 7 and 8 featuring an adequate power, which is responsible for the movement of a respective plunger 9 and 10.

The motors 7 and 8 are connected to the control unit 2 by means of a wired or wireless connection.

The syringe 5 is associated to a pressure sensor 9, so as to remotely detect in real time the pressure present inside the balloon. The pressure sensor 11 is connected to the control unit 2 by means of a wired or wireless connection, as well.

Finally, the assembly 1 comprises an emergency stop button, which is schematically shown in the figure and is indicated with number 12, to be activated by the medical personnel in the event that operating faults occur. The emergency stop button 12 is connected to the control unit 2 by means of a wired or wireless connection, as well.

Both the syringe 5 and the injector 6 comprise limit stop detecting sensors, which allow a reset to be performed at the beginning of the operating cycle and, at the same time, allow the medical personnel to make sure that the assembly correctly works before using it on the patient.

In use, the doctor, after having duly prepared the syringe 5 for the inflation of the balloon inside the channel that has been previously created in the vertebra, remotely activates the movement of the plunger 9 by controlling the stepper motor 7 through the control unit 2.

In particular, the keyboard 4 is arranged behind a protective shield (which is not illustrated for simplicity reasons), so as to protect the doctor from radiations.

The doctor can visually follow the development of the procure by means of the radiography monitoring system normally used and, in so doing, can increase or decrease the pressure inside the balloon and check the results in real time.

The inflation of the balloon can be scheduled and then activated, or it can be controlled in real time by the doctor.

The pressure inside the balloon is constantly detected by the pressure sensor 11 and displayed on the display 3. To this regard, an inflation stop command can be insert, which allows the inflation to be stopped when a given pressure, which has been previously set, is reached.

After the balloon inflation step has ended, the doctor manually removes the syringe 5. During this step, the radiography monitoring system is not active and, therefore, there are no radiations. After having removed the syringe 5, the doctor introduces the injector 6, which is suited to insert the cement.

At this point, the doctor acts in the same way as he acted during the inflation step and stands behind the protective shield. In this way, the doctor remotely activates the movement of the plunger by controlling the stepper motor 8 through the control unit 2.

Also in this case, the insertion of the cement can be scheduled and then activated, or it can be controlled in real time by the doctor.

Therefore, the doctor can remotely perform the insertion of the cement into the cavity by monitoring the process by means of the radiography monitoring system, until the cavity is filled.

At this point, the procedure can be considered as concluded and, after the monitoring system has been disabled, the doctor can safely remove the entire apparatus from the patient.

Obviously, all the devices that come into contact with the patient, such as the syringe, have to be considered as disposable and, therefore, are thrown away at the end of the procedure.

1-7. (canceled)

8. An assembly for kyphoplasty procedures comprising a syringe, which is suited to inflate a balloon inserted into a cavity obtained in the vertebral column, and an injector, which is suited to introduce cement into the vertebral cavity formed, said assembly further comprising a control unit and a pair of motors, each of which is suited to move a respective plunger belonging to the syringe and to the injector respectively, said motors being connected to said control unit.

9. An assembly for kyphoplasty procedures according to claim 8, wherein said motors are stepper motors.

10. An assembly for kyphoplasty procedures according to claim 8, further comprising a pressure sensor, which is connected to said syringe so as to remotely detect in real time the pressure present inside the balloon.

11. An assembly for kyphoplasty procedures according to claim 10, wherein said pressure sensor is connected to the control unit.

12. An assembly for kyphoplasty procedures according to claim 8, further comprising an emergency stop button, which is connected to the control unit.

13. An assembly for kyphoplasty procedures according to claim 8, wherein said control unit comprises displaying means and setting means.

14. An assembly for kyphoplasty procedures according to claim 8, wherein the syringe and the injector comprise limit stop detecting sensors.
